(19) United States
(12) Patent Application Publication
(10) Pub. No.: US 2025/0195624 A1
(43) Pub. Date: Jun. 19, 2025

(54) USE OF SUPEROXIDE DISMUTASE FOR THE TREATMENT AND/OR PREVENTION OF ALLERGIC ASTHMA

(71) Applicant: ISOCELL S.A.S., Paris (FR)

(72) Inventor: Laurent Intes, Paris (FR)

(21) Appl. No.: 18/838,671

(22) PCT Filed: Feb. 21, 2022
(86) PCT No.: PCT/IB2022/000094

§ 371 Date: Aug. 15, 2024

(51) Int. Cl. A61K 38/44; A61K 31/137; A61K 36/42; A61K 36/899; A61K 38/16; A61P 11/06; A61P 37/08

(52) U.S. Cl. CPC A61K 38/446; A61K 31/137; A61K 36/42; A61K 36/899; A61K 38/168; A61P 11/06; A61P 37/08; C12Y 115/01001

(57) ABSTRACT

Use of a superoxide dismutase for the treatment and/or prevention of allergic asthma in mammals, I which the superoxide dismutase is administered orally alone, or optionally in combination with the administration of an aerosol preparation comprising at least one β₂-adrenergic receptor agonist.

The present invention relates to the use of superoxide dismutase in the treatment and/or prevention of allergic asthma.

The name superoxide dismutase (E.Cl.15.1.1.) otherwise known as SOD for short, includes a family of ubiquitous metalloenzymes implicated in the dismutation of the superoxide (02) anion. The accumulation, or too great a production, in vivo, of the superoxide radical species is harmful for most living organisms. A physiological state in which this has occurred is known as oxidative stress, and is associated with a number of metabolic disorders, that are themselves involved in various pathological processes such as carcinogenesis, atherosclerosis, ageing and inflammatory disorders, such as coeliac disease. Apart from the degree of evolution or cellular localisation, SOD is present in three main forms that are distinguished by the metal ions contained within the molecule, that is to say copper-zinc or CuZn-SOD, manganese or Mn-SOD and iron or Fe-SOD. CuZn-SOD and Mn-SOD are also to be found in the more specialized cellular structures such as the peroxisomes or the chloroplasts in plants, whereas in mammals, an extracellular CuZn-SOD or Ec-SOD has been specifically found in the extra-cellular compartment. Many experiments have shown that even if all of the SODs have the same basic anti-radical activity, their effective biological activity is not identical with respect to a given therapeutic indication and target organism. This has also been shown to be true in regard to the difference in activity between homologous and heterologous SODs. In the present specification, the expression “homologous SOD” refers to a SOD produced natively by the cell of a species under investigation. The expression “heterologous SOD” refers to a SOD of foreign origin, i.e. produced by, or coming from a species which is different to the species under investigation. For example, the anti-inflammatory activity of homologous SODs in foot oedema induced by carrageenan or adriamycin in the rat is non-functional compared to that of SODs of heterologous origin. The effectiveness of heterologous SODs seems to depend on variations, even subtle ones, in the amino acids of the enzymes rather than in the type of metal present at the active site or the global molecular mass of the enzyme.

A common way of administering SODs is orally, often prepared as a solid dosage form preparation, and presented as a tablet or an ingestible capsule. However, oral administration of SODs can be problematic in that SODs are often rapidly degraded in the gastrointestinal tract, thus leading to reduced bioavailability, and efficacy. This problem is compounded by the difficulty of getting the SODs to the particular cell location where they will be the most effective. One known commercial development which addresses the problem of the in vivo digestive stability of orally administered SOD is the solid dosage form product identified as Glisodin® (Isocell, France), which includes a plant-derived SOD in association with a prolamine obtained from wheat, and in particular the prolamine known as gliadin. Glisodin® is currently known and marketed as a dietary supplement for people suffering from skin depigmentation issues such as vitiligo, for general overall human body wellbeing, and as an anti-ageing skin treatment and beautifier. A number of other studies have also reported the oral administration of Glisodin® for:
inhibiting oxidative stress:
Vouldoukis et al., “Supplementation with gliadin-combined plant superoxide dismutase extract promotes antioxidant defences and protects against oxidative stress”, Phytotherapy Research, 2004, 18 (12), 957-962;
Nakajima et al., “Oral supplementation with melon superoxide dismutase extract promotes antioxidant defences in the brain and prevents stress-induced impairment of spatial memory”, published in Behavioural Brain Research, 2009, 200 (1), 15-21;
C. Muth et al., “Influence of an orally effective SOD on hyperbaric, oxygen related cell damage” University Medical School, Ulm, Free Radical Research, 2004;
inhibiting ultraviolet oxidative stress: Mac-Mary et al, “Could a photobiological test be a suitable method to assess the anti-oxidant effect of a nutritional supplement (GliSODin)?”, European Journal of Dermatology, 2007, 17 (3), 254-255);
promoting immune modulation: Okada et al., “Prevention of inflammation-mediated acquisition of metastatic properties of benign mouse fibrosarcoma cells by administration of an orally available superoxide dismutase”, British Journal of Cancer, 2006, 94 (6), 854-862);
inhibiting vascular inflammation: M. Cloarec, and al., “GliSODin, a vegetal SOD with Gliadin, as preventative agent vs. atherosclerosis, as confirmed with carotid Ultrasound-B Imaging” American Hospital, Paris, European Annuals of Allergy & Clinical Immunology, 2007;
   Asthma is a chronic pathology affecting the lower respiratory tract, the symptoms of which are displayed by inflammation, broncho-constriction and bronchial hypersecretion. Throughout the world, more than 300 million people are considered to be asthmatic, and an estimated 70% of those people are estimated to suffer from an allergic form of asthma.

Allergic asthma is characterised by an abnormal reaction of the immune system to inhaled particles, such as those formed by dust mites, animal detritus and desquamations, fungal spores, and plant particles, e.g. pollen. In the majority of the population, these particles are recognised by, but also tolerated by, the immune system, and as such do not production any untoward reaction. However, in affected subjects, these particles induce a T-helper 2 cell (Th2) immune response and the subsequent production of IgE immunoglobulins, which in turn leads to basophilic and mastocyte degranulation, thereby releasing a number of inflammatory response mediators such as histamine and leukotrienes. These mediators lead to broncho-constriction and form the basis of an asthma attack. However, it is also known that other populations of T helper cells are implicated in asthma, such as T helper 17 cells or T-regulators. These various immunological mechanisms lead to tissue remodelling of the airway in humans, along with an excess production of mucus, and contraction of the smooth muscles structures of the respiratory pathway.

It is also known that there isn't just a single type of asthma, but that numerous phenotypes exist in humans, however two main phenotypes can be distinguished: Th2-high and Th2-low. Th2-high phenotypes are characterised by a high level of serum IgE and a large proportion of eosinophils. Th2-low phenotypes involve other mediators, such as Th17 cells. Current therapies, such as those using corticosteroids for non-severe forms of asthma, or anti-IgE treatments, such as the monoclonal antibody omalizumab, which has been available for a decade or so, or more recently the anti-IL-5 monoclonal antibody reslizumab are able, on the whole, to manage these symptoms. These products need to be administered intravenously, however, and have a number of side-effects which can be problematic, including: cough; difficulty with swallowing; dizziness; faintness; lightheadedness when getting up suddenly from a lying or sitting position; fast heartbeat; hives, itching, or skin rash; puffiness or swelling of the eyelids or around the eyes, face, lips, or tongue; redness of the skin; tightness in the chest; and unusual tiredness or weakness.

One aspect of the present invention is therefore to provide another, less invasive, and less problematic treatment for allergic asthma, that doesn't have the drawbacks associated with intravenous administration of other contemporary therapeutic treatments, and which can optionally and additionally be associated, if so desired, with more classical treatment regimes for managing the asthmatic condition.

According to one aspect therefore, the invention relates to a use of a superoxide dismutase for the treatment and/or prevention of allergic asthma in mammals, wherein the superoxide dismutase is administered orally alone, or optionally in combination with the administration of an aerosol preparation comprising at least one ß₂-adrenergic receptor agonist.

According to yet another aspect, the use of a superoxide dismutase for the treatment and/or prevention of allergic asthma relates to a use where the mammal is a human.

The superoxide dismutase can be chosen from those generally known to the skilled person, and may be chosen from the group consisting of human superoxide dismutases, animal superoxide dismutases, bacterial superoxide dismutases, yeast superoxide dismutases and plant superoxide dismutases. Advantageously however, the at least one superoxide dismutase is selected from the group consisting of CuZn superoxide dismutases, Mn superoxide dismutases, extra-cellular superoxide dismutases, Ni superoxide dismutases, and Fe superoxide dismutases. According to one aspect, the superoxide dismutase is a homologous or a heterologous superoxide dismutase.

According to yet another advantageous aspect, a heterologous superoxide dismutase is preferred.

According to a particularly advantageous aspect, the superoxide dismutase is a plant-derived superoxide dismutase, and even more advantageously a heterologous plant superoxide dismutase. Such a superoxide dismutase can be obtained or produced in many different ways. For example, the at least one superoxide dismutase can be extracted from plants. If plants are used for the extraction or production of the at least one superoxide dismutase, such plants are helpfully members selected from the group consisting of the Cucurbitaceae, Solanaceae, and Triticum species of plants, and/or their various cultivars, and are advantageously selected from the group consisting of melon, e.g. Cucumis melo, tomato, e.g. Lycopersicum esculentum, and wheat, e.g. Triticum aestivum, Triticum vulgare, and Triticum durum, and the like.

Among the various plant superoxide dismutases available, and according to another aspect, the superoxide dismutase is selected from the group of plant superoxide dismutases consisting of peroxisomal, chloroplastic and cytosolic superoxide dismutases.

According to yet another aspect, the superoxide dismutase is a plant-derived superoxide dismutase selected from the group consisting of melon superoxide dismutase, and wheat superoxide dismutase.

Extraction techniques for such plant-derived superoxide dismutases are well known per se, and the corresponding dried extracts, for example, of melon or wheat, are available commercially. For example, with regard to a superoxide dismutase obtained from Cucumis melo L varieties, the superoxide dismutase is generally provided in powder form as a freeze dried extract of melon juice, and commercially available under the trade name “SOD B®” from Bionov, France. Such a powder is generally light orange in colour, with a measured SOD enzymatic activity measured of 90,000 U (NBT)/g-150,000 U (NBT)/g. Similarly, dried extract of wheat SOD from Triticum vulgare plants is commercially available from Silab, under the INCI name “Triticum Vulgare (Wheat) Germ Extract”, CAS No 84012-44-2, said extract presenting as a beige-coloured lyophilized powder with a SOD enzymatic activity of 1,500,000 U (NBT)/g-3,000,000 U (NBT)/g. Where expressed in the present specification, the enzymatic activity of SOD is expressed in units U (NBT)/g, the determination of the activity being made according to a known NBT analysis protocol, such as that described by Zhou et al, in J. Pharm. Biomed. Anal. 2006 March 18; 40 (5): 1143-8, in Sections 2.1, 2.2.1, 2.3.1, 2.4.2 and 2.4.2.1, the contents of which are incorporated herein by reference.

According to yet another aspect, the oral administration of superoxide dismutase consists of a solid-form preparation comprising a plant-derived superoxide dismutase in association with a prolamine. Advantageously, the prolamine can comprise at least one prolamine based peptide selected from the group consisting of a fragment of gliadin or a derivative, analogue, salt or metabolite thereof. Even more advantageously, the prolamine based peptide fragment is a non-immunogenic analogue of gliadin. In a particularly advantageous aspect, the prolamine based peptide fragment is a non-immunogenic analogue of gliadin having competitive inhibiting activity with respect to immunogenic prolamine based peptides. The at least one prolamine based peptide fragment can also suitably be selected from the group consisting of fully hydrolysed, substantially hydrolysed or slightly hydrolysed prolamine based peptide fragments. In general, however, the prolamine is a prolamine fragment chosen from the group consisting of those fragments obtained from PTC (pancreatin, trypsin, chymotrypsin) hydrolysed prolamine mimicking the processes of gastrointestinal hydrolysis. According to one particularly advantageous aspect, the prolamine is a prolamine fragment which has been hydrolysed to an extent in which it functions as a targeting signal within the intestinal tract.

Advantageously, and according to another aspect, the oral administration of superoxide dismutase consists of administering a solid-form preparation comprising a plant derived superoxide dismutase in association with gliadin. Such a product is known commercially as a solid dosage-form preparation sold under the trade name Glisodin® by Isocell, France.

According to yet another aspect, the oral administration of superoxide dismutase consists of a solid-form preparation comprising:
from 0.50% to 5.00%, by total weight of the solid form preparation, of a dried melon juice-derived extract containing a standardised titre of superoxide dismutase;
a) from 0.50% to 5.00%, by total weight of the solid form preparation, of a dried melon juice-derived extract containing a standardised titre of superoxide dismutase;
b) from 1.50% to 5.00%, by total weight of the preparation, of gliadin;
c) qsp maltodextrin,
d) wherein the superoxide dismutase has an enzymatic activity comprised between 1 to 5 U (NBT)/mg.

According to another advantageous aspect, the oral administration of superoxide dismutase consists of a solid-form preparation consisting of:
a) 1.33% by total weight of the solid form preparation, of a dried melon juice-derived extract containing a standardised titre of superoxide dismutase;
b) 3.33%, by total weight of the preparation, of gliadin;
c) qsp maltodextrin,
d) wherein the superoxide dismutase has an enzymatic activity of 1.5 U (NBT)/mg.

According to another aspect, the oral administration of superoxide dismutase consists of a solid-form preparation comprising:
a) from 0.04% to 0.40%, by total weight of the preparation, of a wheat-derived superoxide dismutase;
b) from 1.50% to 5.00%, by total weight of the preparation, of gliadin;
c) qsp maltodextrin,
d) wherein the superoxide dismutase has an enzymatic activity comprised between 1 to 10 U (NBT)/mg.

According to still yet another aspect, the oral administration of superoxide dismutase consists of a solid-form preparation comprising:
a) 0.18%, by total weight of the preparation, of a wheat-derived superoxide dismutase;
b) 3.50%, by total weight of the preparation, of gliadin;
c) qsp maltodextrin,
d) wherein the superoxide dismutase has an enzymatic activity of 4.5 U (NBT)/mg.

According to yet further aspect, the superoxide dismutase is administered orally to a human in an amount comprised between 100 mg to 2000 mg per day, continuously for a minimum period of 15 days.

According to yet another advantageous aspect, the superoxide dismutase is administered orally to a human in an amount of 500 mg per day, continuously for a minimum period of 15 days, and up to 180 days.

As has been indicated above, according to another aspect, the superoxide dismutase is administered orally, but in combination with, the administration of an aerosol preparation comprising at least one ß₂-adrenergic receptor agonist. The short-term treatment of allergic asthma using ß₂-adrenergic receptor agonists is known per se. According therefore to another aspect, the at least one ß₂-adrenergic receptor agonist is a short-acting ß₂-adrenergic receptor agonist, also known as SABA for short, selected from the group consisting of bitolterol, fenoterol, isoprenaline, levosalbutamol, orciprenaline, pirbuterol, procaterol, ritodrine, salbutamol, and terbutaline. According to another advantageous aspect, the at least one ß₂-adrenergic receptor agonist is salbutamol.

### BRIEF DESCRIPTION OF THE FIGURES

The present invention will now be described with regard to the following detailed examples, and with reference to the annexed figures, useful for non-limitative illustrative purposes of the various aspects put forward in the present specification

FIG. 1 is a series of charts plotting the results of an in vitro mixed lymphocyte reaction analysis in naïve control mice and asthmatic mice, in the presence or absence of a Glisodin formulation as described herein;

FIG. 2 is a series of charts plotting the results of lymphocyte reactivation and proliferation in an allergic asthma adoptive transfer model, in the presence or absence of a Glisodin formulation as described herein;

FIG. 3 is a series of charts plotting the results of the analysis of the subpopulation of CD4+ T-lymphocytes that had proliferated from the in adoptive transfer tests for which the results are illustrated in FIG. 2;

FIG. 4 is a series of charts plotting total immunoglobulin production induced by house dust mite (HDM) allergic conditions in mice, in the presence or absence of a Glisodin formulation as described herein;

FIG. 5 is a series of charts plotting allergy specific immunoglobulin production induced by house dust mite (HDM) allergic conditions in mice, in the presence or absence of a Glisodin formulation as described herein;

FIG. 6 is a series of charts plotting chemoattractant analysis in house dust mite (HDM) allergy induced mice, in the presence or absence of a Glisodin formulation as described herein.

FIG. 7 is a series of charts plotting respiratory function in house dust mite (HDM) allergy induced mice, in the presence or absence of a Glisodin formulation as described herein;

FIG. 8 is a series of charts plotting respiratory function in house dust mite (HDM) allergy induced mice, in the presence or absence of a Glisodin formulation as described herein, with or without co-administration of salbutamol;

FIG. 9 is a series of charts plotting cell population from bronchoalveolar lavages (BAL) analysed using flow cytometry in house dust mite (HDM) allergy induced mice, in the presence or absence of a Glisodin formulation as described herein, with or without co-administration of salbutamol;

FIG. 10 is a series of charts plotting immune response in house dust mite (HDM) allergy induced mice, in the presence or absence of a Glisodin formulation as described herein, with or without co-administration of salbutamol;

FIG. 11 is a series of charts plotting serum levels of IgE in house dust mite (HDM) allergy induced mice, in the presence or absence of a Glisodin formulation as described herein, with or without co-administration of salbutamol;

FIG. 12 is a series of charts plotting cytokine response in house dust mite (HDM) allergy induced mice, in the presence or absence of a Glisodin formulation as described herein, with or without co-administration of salbutamol;

FIG. 13 is a series of histological stains of lung samples taken from house dust mite (HDM) allergy induced mice, in the presence or absence of a Glisodin formulation as described herein, with or without co-administration of salbutamol.

### DETAILED DESCRIPTION

A number of experiments were carried out to demonstrate the effect of administration of a plant-derived SOD in mammals. For the purposes of the experimental evaluation, the following Glisodin® formulations were prepared:

TABLE 1

| Ingredient | Wheat Glisodin | Melon Glisodin |
|---|---|---|
| Standardised SOD-titred freeze- | 0 | 1.33% |
| dried melon juice extract | | |
| Standardised SOD-titred freeze- | 0.18% | 0 |
| dried wheat germ extract | | |
| Gliadin | 3.50% | 3.33% |
| Maltodextrin | 96.32% | 95.34% |
| SOD activity (units of NBT/mg) | 4.5 | 1.5 |

### Experimental Protocols

The experimental evaluations focussed on the following areas:
1) impact of the Glisodin® formula in vitro on lymphocyte reactivation in an allergic asthma model, also known as a mixed lymphocyte reaction or MLR;
2) impact of Glisodin® formula in vivo on lymphocyte reactivation in an allergic asthma model, also known as an adoptive transfer model or AT;
3) impact of the Glisodin® formula on the humoral response and production of chemoattractant molecules, on a molecular scale;
4) analysis of the respiratory function of the mice having received the Glisodin® formula, and/or having been treated with salbutamol;
5) analysis of any anti-inflammatory effect on the treated mice with either Glisodin® and/or salbutamol;
6) analysis of the comparative immune response of treated mice with either Glisodin® and/or salbutamol;
7) comparative analysis of the humoral response between mice treated with Glisodin® and/or salbutamol;
8) comparative analysis of the cytokine response between mice treated with Glisodin® and/or salbutamol;
9) comparative histological analysis between mice treated with Glisodin® and/or salbutamol.

An allergic asthma murine model involving exposure to house dust mites was used. This model comprises a preliminary percutaneous sensitisation challenge phase of Balb/cj mice with a whole house dust mite extract (days 0, 7, 14, and 21), followed by two intranasal stimulations (days 27 and 34) which induced the symptoms of allergy-induced asthma in the test subjects. The efficacy of the Glisodin formulations was evaluated by daily administration to the mice via force feeding of 5 mg of the Glisodin formulation diluted in water, starting at 24 hours after the first intranasal sensitisation challenge and continuing for 5 consecutive days every week for 5 weeks. For comparative purposes, the same protocol was used to evaluate the effect of a combined administration of Glisodin with salbutamol, in which case the salbutamol was administered for 30 seconds at a solution concentration of 5 mg/ml as a nebulized aerosol 1 hour before each sensitisation challenge.

### Part 1—In Vitro Mixed Lymphocyte Reaction (MLR)

For part 1 of the analyses, i.e. in vitro MLR, dendritic cells from 6-week old Balb/cj mice were caused to differentiate from tibial bone marrow stem cells, i.e. bone marrow derived cells, also known as BMDC. Similarly, CD4+ and CD8+ (LT) lymphocytes were isolated from the spleen of these same mice and enriched using negative selection. The lymphocytes obtained from the asthmatic mice were reactivated using dendritic cells loaded with allergen, i.e. with house dust mites. In order to measure the impact of the Glisodin® formula on the interaction between T-lymphocytes and BMDC, a co-culture of BMDC and LT was carried out in the presence or absence of the Glisodin® formula. After co-culture, the T-lymphocytes underwent phenotypical analysis via flow cytometry using the following markers: IFN-FITC, Foxp3-Pe, CD4-PercP5.5, IL-4-APC, CD8-APC-H7, IL-17-Brilliant Violet 421, and CD25-Brilliant Violet 510.

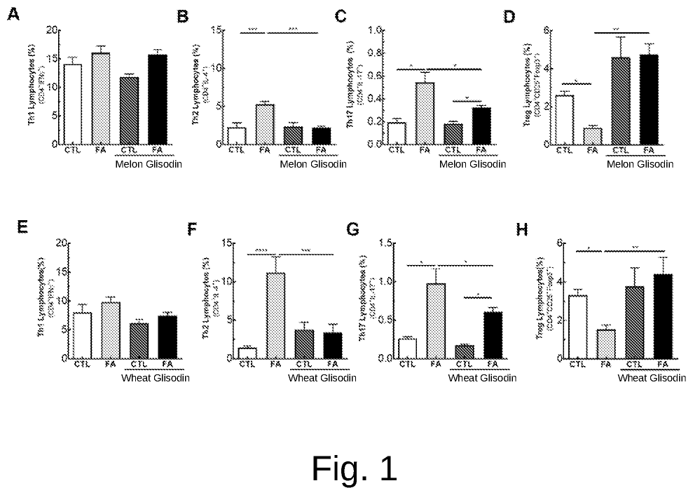

The results are presented in FIG. 1. Lymphocyte differentiation from naïve mice (CTL=control, white and dark grey) or asthmatic mice (FA, light grey and black histograms) was measured from autologous dendritic cells loaded with human dust mite in the presence or absence of melon Glisodin (cf. FIGS. 1A and 1D) or wheat Glisodin (cf. FIGS. 1E and 1H). CD4+ lymphocyte differentiation was also measured via population identification: Th1 (FIGS. 1A and 1E), Th2 (FIGS. 1B and 1F), Th17 (FIGS. 1C and 1G) and Treg (FIGS. 1D and 1H). Application of ANOVA analysis using the Tukey test gave a confidence interval as marked in the figure: *=p<0.05, **=p<0.01, and ***=p<0.001, for a mouse population of n=6 to 8 subjects per group.

With regard to Th1 lymphocytes characterised by the secretion of γ-interferon (γ-IFN), no modification of the differentiation, when comparing cells from naïve mice to asthmatic mice, was observed, nor was there any noticeable impact of the presence or absence of the Glisodin formulations (cf. FIGS. 1A and 1E), the profiles remaining ostensibly the same. These results were to be expected given that the Th1 response is mainly implicated in the antibacterial and antiviral immune response, and is relatively little involved in the allergic response. In opposition to this, Th2 lymphocytes are implicated mainly in the antiparasitic and anti-allergic response. We observed a noticeable differentiation of Th2 lymphocytes from asthmatic mice in comparison to naïve mice, with an increase in differentiation of the order of 100 to 700% (cf. FIGS. 1B and 1F, white and light grey histograms). Of particular interest here is that the differentiation is not observed when the cells are in the presence of the Glisodin formulation, suggesting an inhibition of allergy induced Th2 differentiation by the Glisodin formulation (cf. FIGS. 1B and 1F, dark grey and black histograms). Furthermore, the presence of the Glisodin formulation has no effect on the differentiation of Th2 lymphocytes in naïve mice. Similarly, Th17 lymphocyte differentiation, which is implicated in the inflammatory response, is increased by a factor of 3 to 5 with asthmatic mice compared to naïve mice (cf. FIGS. 1C and 1G). Of particular note here too is the fact that the presence of either wheat Glisodin or melon Glisodin also inhibits Th17 lymphocyte differentiation, although not to the extent seen naïve mice (cf. FIGS. 1C and 1G). It should also be noted that these lymphocyte responses are under regulation by T regulator lymphocytes (Treg), their differentiation being characterised by a high expression of CD25 and transcription factor FoxP3 (cf. FIGS. 1D and 1H). As would be expected, a drop in the number of differentiated Treg in asthmatic mice, compared to naïve mice is observed. Nonetheless, even here, the presence of Glisodin increases the frequency of Treg differentiation in comparison to asthmatic mice, in both naïve and asthmatic mice (cf. FIGS. 1D and 1H). In summary, these results demonstrate that both wheat and melon Glisodin don't seem to have an impact on Th1 differentiation, but do inhibit both Th2 and Th17 lymphocyte differentiation in an allergic environment. In opposition to this, the presence of Glisodin can be seen to increase differentiation in Treg lymphocytes, irrespective of the presence or absence of an allergic environment.

Further analysis involved determining CD8+ lymphocyte activation by measuring their capacity to produce y-IFN, which is known to be capable of maintaining pulmonary inflammation in allergy induced asthma. The results obtained showed that there were no statistically significant differences between the two types of mice, nor were there any significant differences in the type of plant-derived SOD used in the Glisodin formulation. These results suggest an absence of an effect for Glisodin with regard to CD8+T-lymphocytes, in contrast to the effect observed with CD4+T-lymphocytes.

### Part 2—In Vivo Adoptive Transfer

For part 2 of the analyses, i.e. in vivo adoptive transfer (AT), T-lymphocyte CD4+ and CD8+ cells are isolated from the spleen of naïve or asthmatic Balb/cj Ly5.1 mice, and enriched via negative selection. In order to determine the effect of the Glisodin® formula on the reactivation of the T-lymphocytes during the allergic reaction, isolated T-lymphocytes are marked with carboxyfluorescein succinimidyl ester (CFSE) in order to measure their proliferation, and then reinjected into naïve mice intravenously at a level of 2-3 million cells. Following on from this, the mice are exposed twice to house dust mites, in order to reactivate the injected cells in vivo, which mice have also either received an administration of the Glisodin® formula above, or not, for comparison. After T-lymphocyte reactivation, the latter are analysed for proliferation and phenotype in the lungs and spleen of the mice using the following flow cytometry markers: CFSE, CD122-FITC, Ly5.1-Pe, CD4-PercP5.5, CCR4-Pe-Cy7, CCR6-Pe-Cy7, GATA3-APC, RORyt-APC, CD8-APC-H7, IL-17-Brilliant Violet 421, CD44-Brilliant Violet 421, CXCR3-Brilliant Violet 510 and IL-17RA-Brilliant Violet 510.

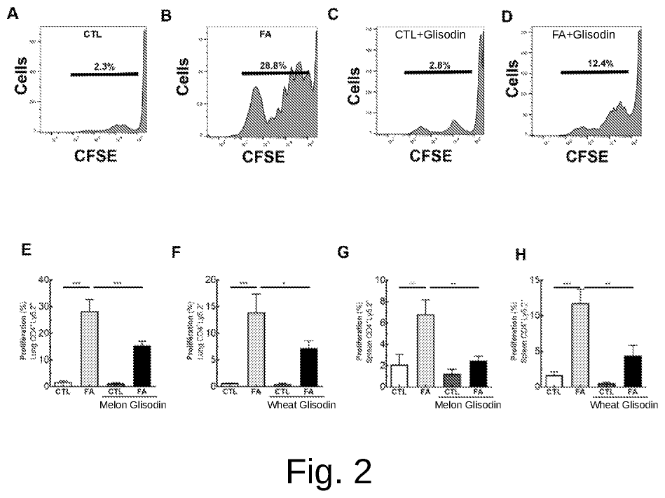

Lymphocyte proliferation was measured following exposure to the house dust mites, the results of which are presented in FIG. 2. This measurement was obtained by analysing the progressive dilution of CFSE, the results of which are shown in FIGS. 2A to 2D with regard to proliferation in lung and spleen cells, with FIGS. 2E to 2H showing proliferation percentage in lungs (FIGS. 2E and 2F) and spleen (FIGS. 2G and 2H) respectively. Application of ANOVA analysis using the Tukey test, gave a confidence interval as marked in the figure: *=p<0.05, **=p<0.01 and ***=p<0.001, with a population of n=6 to 8 mice per group.

A marked increase in T-lymphocyte proliferation was observed for the transferred T-lymphocytes, using a Ly5.2 marker, for the asthmatic mice (light grey histograms), in comparison to those lymphocytes from naïve mice (white histograms), which did not show signs of proliferation (cf. FIGS. 2E to 2H). The observed proliferation is therefore a marker for the reactivation of lymphocytes which have become sensitised post-exposure to the house dust mites. Similarly, an increase in the reactivation of lymphocytes transferred from asthmatic mice to the mice that were also exposed to Glisodin (black histograms), was also observed, in comparison to those lymphocytes from naïve mice (dark grey histograms). Of particular interest, however, is the observation that lymphocyte reactivation was reduced by approximately 50% in mice that were also exposed to the Glisodin formulations, in comparison to mice not exposed to the Glisodin formulations. No significant difference in activity was observed between the wheat SOD and melon SOD Glisodin formulations.

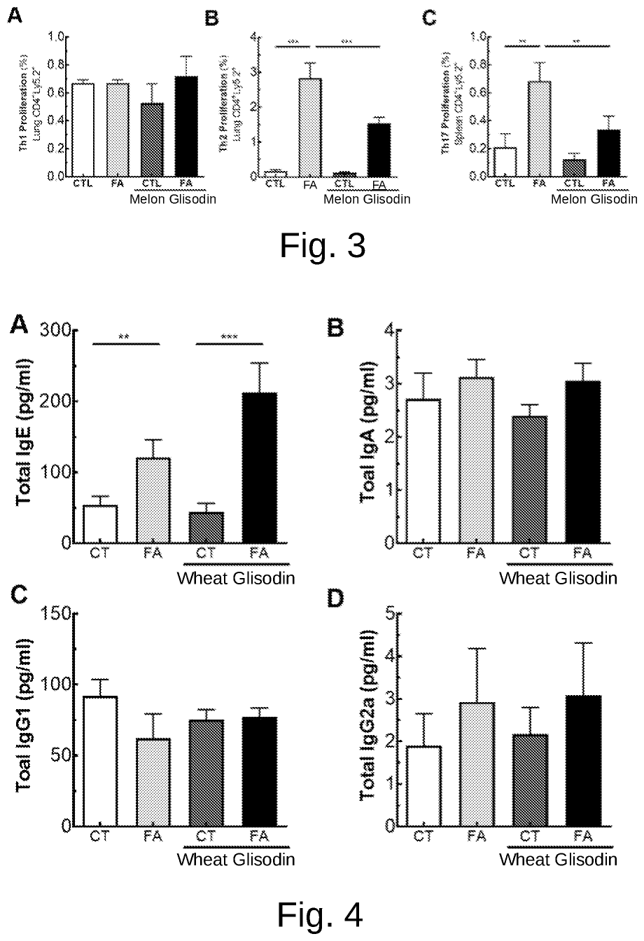

Further analysis was carried out here to determine the subpopulation of CD4+T-lymphocytes that had proliferated, the results of which are shown in FIG. 3. This was achieved by carrying out the population analysis amongst those cells obtained from the donor that had proliferated, using Ly5.2 marker and CFSE dilution. It was observed that the frequency of Th1 lymphocytes was not affected, whether through an allergy-induced environment or via application of Glisodin, as illustrated in FIG. 3A. Nonetheless, an increase in the proliferation of Th2 and Th17 lymphocytes was observed in allergic mice, in comparison to the control mice, (cf. FIGS. 3B and 3C). Additionally, as confirmation of the in vitro results, it can be seen from the in vivo results that the Glisodin formulations inhibit cellular proliferation of both Th2 and Th17 lymphocytes which are induced through allergy, (cf. FIGS. 3B et 3C). These results confirm an anti-inflammatory and anti-allergenic effect of the Glisodin formulations both in vitro and in vivo.

### Part 3—Molecular Scale Analysis

For part 3 of the experiments, mice serum from part (a) are used. The total, and specific, amounts of the following antibodies directed against the house dust mite were evaluated using ELISA: IgE, IgA, IgG1 and IgG2a. Similarly, bronchoalveolar lavage (BAL) from part (2) were used to determine the quantities of the following chemoattractant and pro-inflammatory molecules: RANTES or CCL5, a known chemotactic molecule for T-cells, eosinophils and basophils, which play an active role in the recruitment of leukocytes at sites of inflammation, Eotaxin or CCL11, a known chemotactic molecule for eosinophils, KC or keratinocyte-derived chemokine or CXCL1, a known chemotactic molecule for neutrophils, MCP-1 or CCL2, a known chemotactic molecule for monocytes, lymphocytes as well as for basophils for which they cause degranulation, MIP1 or CCL3, which is implicated in the acute inflammatory state in the recruitment and activation of polymorphonuclear leukocytes, and TARC or CCL17, a powerful chemokine which attracts regulatory T-cells. These determinations were made across 42 samples, as follows:
7 controls with the wheat Glisodin formulation;
8 controls with the melon Glisodin formulation;
9 asthmatic test samples with wheat Glisodin;
8 asthmatic test samples with melon Glisodin;
10 asthmatic test samples as the positive control.

First, the total quantity of antibodies produced was determined from mouse sera. This measurement was carried out by ELISA, with application of an ANOVA analysis, giving a confidence interval as marked: *=p<0.05, **=p<0.01 and ***=p<0.001, and a population of n=6 to 8 mice per group.

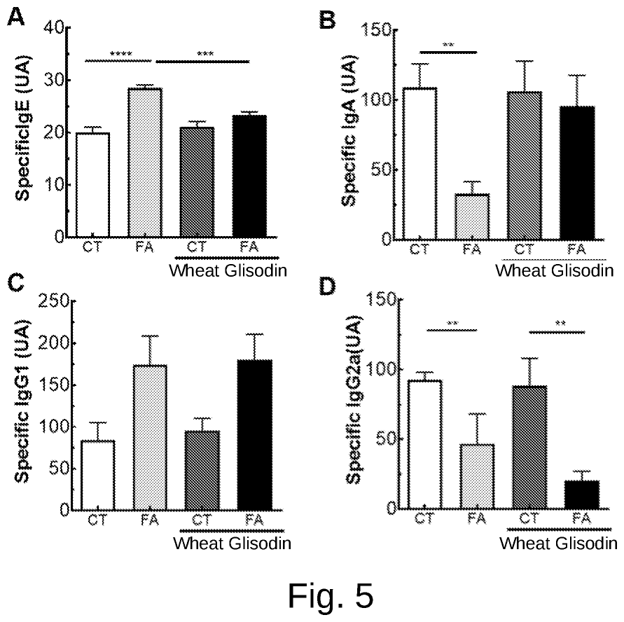

An increase in the total number of IgE was observed in allergic mice, in comparison to the control mice (cf. FIG. 4A). However, no noticeable effect on total IgE production was observed in either the presence or the absence of the Glisodin formulation, in either control or allergy induced asthmatic mice, (cf. FIG. 4A). Furthermore, no modulation in the production of IgA, IgG1 or IgG2a was observed, irrespective of the type of mouse under investigation, and irrespective of the presence or absence of the Glisodin formulations (cf. FIGS. 4B to 4D). Similar results were obtained with the melon Glisodin formulation (results not shown). In order to try and better understand these observations, house dust mite (HDM) specific Igs were measured, the results of which are illustrated in FIG. 5, with application of an ANOVA analysis using the Tukey test, giving a confidence interval as marked: *=p<0.05, **=p<0.01 and ***=p<0.001, with a population of n=6 to 8 mice per group.

In contrast to what was observed with regard to total Ig, an increase in IgE specific to house dust mite (HDM) s in asthmatic mice was observed, in comparison to the control mice (cf. FIG. 5A). The presence of the Glisodin formulations had no noticeable effect on the naïve control mice, but was found to inhibit the increase in the specific IgE which were induced in the allergic asthma protocol (cf. FIG. 5A). In contrast, the house dust mite (HDM) specific IgA are reduced in the allergic subjects, and the Glisodin formulations also inhibited this reduction (cf. FIG. 5B), in comparison to the control mice. No Glisodin effect was observable with regard to reducing the production of house dust mite (HDM) specific Ig2a and IgG1 (cf. FIGS. 5C and 5D). Taken together, these results demonstrate that the Glisodin formulations provide a protective effect against allergic reactions centred on the reduction in specific IgE, and an increase in the specific IgA.

Similar results were obtained with the melon Glisodin formulations (not shown). The SODs contained in these formulations are therefore useful in the treatment and or prevention of allergic asthma.

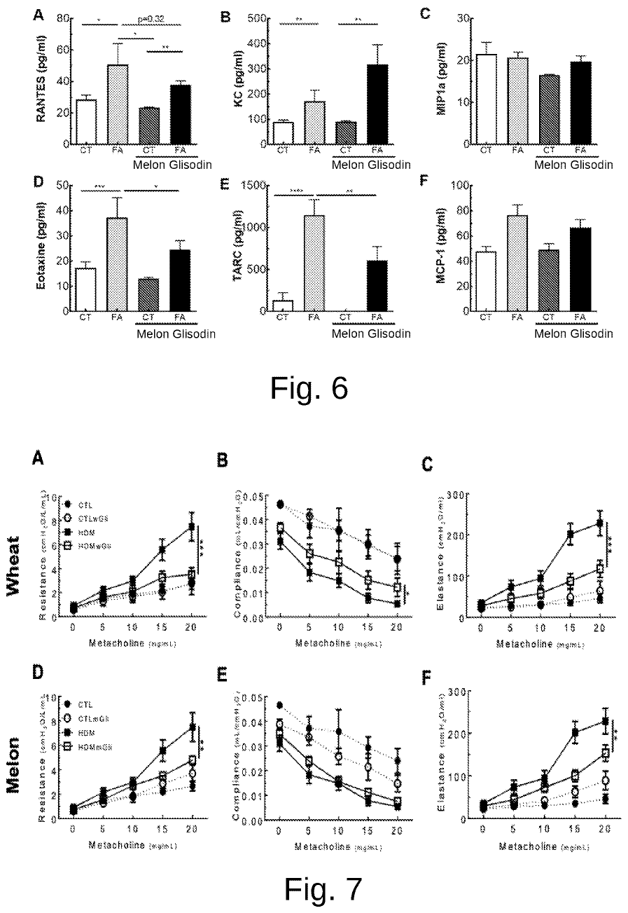

Similarly, bronchoalveolar lavage (BAL) from the previous experiment in mice which had been exposed to the Glisodin formulations were further analysed for the presence and quantity of certain chemoattractants, i.e. RANTES, KC, MCP-1, MIP1, TARC, Eotaxin as illustrated in FIG. 6, and for which an ANOVA analysis using the Tukey test was carried out, giving a confidence interval as marked: *=p<0.05, **=p<0.01 et ***=p<0.001 with a population n=6 to 8 mice per group. The results show an increase in the production of RANTES, KC, Eotaxin and TARC in the allergic mice, in comparison to the control mice (cf. FIGS. 6A, 6B, 6D and 6E), although this increase is not observed for MIP1 and MCP1 (cf. FIGS. 6C and 6F). Of particular interest here is that the mice that were supplemented with the Glisodin formulations showed an increased inhibition in the production of those chemoattractants RANTES, Eotaxin and TARC, the production of which is induced by allergic asthma (cf. FIGS. 6A, 6D and 6E). Similarly, the Glisodin formulations had no observable effect on KC, MIP1 or MCP1 (cf. FIGS. 6B, 6C and 6F).

### Part 4—Respiratory Function

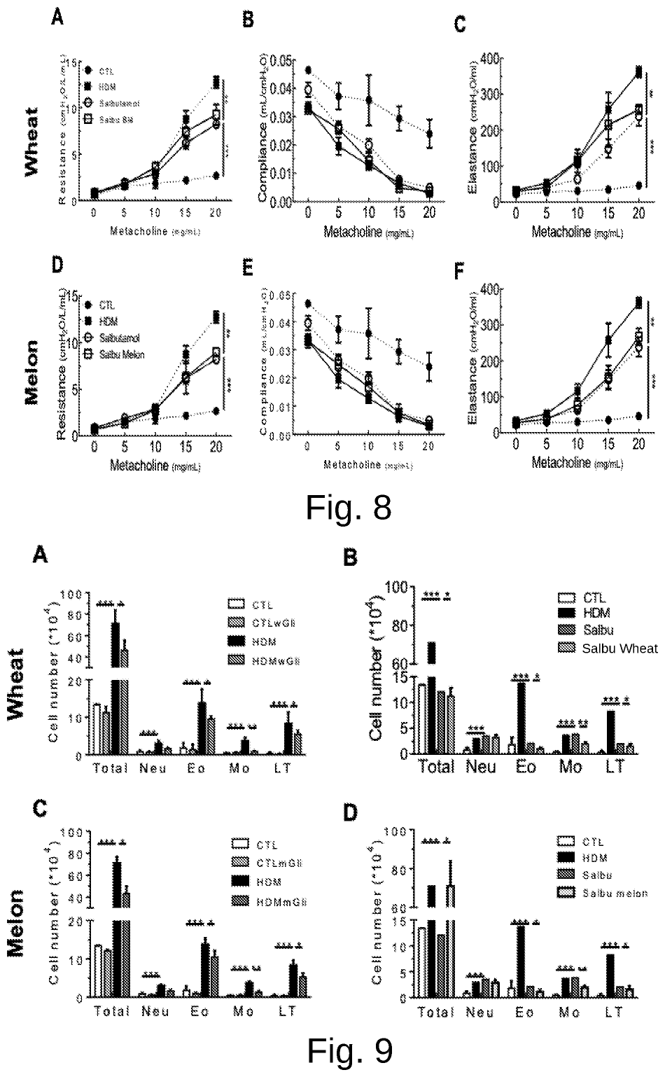

Respiratory function in the mice was analysed using the flexi Vent™ system available from SciReq and the results illustrated in FIGS. 7 and 8. This showed the absence of any effect for Glisodin formulations alone in healthy mice, and a significant improvement in the respiratory function of asthmatic mice. The effect can be seen as a reduction in the resistance of the airways, as shown in FIGS. 7A and 7D, which reflects the resistance of the lung as air passes through it, and which resistance increases in asthma-affected lungs due to a narrowing of the bronchial diameter. The improvement in respiratory function procured by Glisodin is also observed in the compliance plots shown in FIGS. 7B and 7E, compliance here being the capability of the lung to adapt its volume in response to a change in pressure, but also to changes in elastance, as illustrated in FIGS. 7C and 7F, which is the inverse of compliance, and which indicates the elasticity of the lung. In the charts of FIG. 7, naïve untreated mice are indicated as the control, or CTL, naïve mice having received wheat Glisodin as CTLwGli, naïve mice having received melon Glisodin as CTLmGli, asthmatic mice as HDM, and asthmatic mice having received either wheat Glisodin (HDMwGli) or melon Glisodin (HDMmGli). Respiratory function was measured using the flexiVent™ system in response to increasing doses of the broncho-constrictor methacholine. ANOVA analysis of the data using the Tukey test was carried out and a confidence interval as indicated on the charts established: *=p<0.05, ** p<0.01 and *** p<0.001, for a population n=6 to 8 mice per group.

The efficacy of Glisodin was also analysed in combination with salbutamol, a B2-adrenergic receptor agonist having a bronchodilatory and anti-inflammatory activity, as used in the treatment of asthma attacks, the results of which are illustrated in FIG. 8. Here, a significant effect on the improvement of respiratory function through the administration of salbutamol was apparent with regard to resistance (cf. FIGS. 8A and 8D), compliance (cf. FIGS. 8B and 8E), and elastance of the airways (cf. FIGS. 8C and 8F). However, no synergistic, or additive, effect could be seen in the combination of both Glisodin administration and salbutamol administration. In the charts, CTL or control indicates naïve untreated, untreated asthmatic mice (HDM), asthmatic mice having received salbutamol treatment (Salbutamol), asthmatic mice having received salbutamol and wheat Glisodin (Salbu Wheat) or asthmatic mice having received salbutamol with melon Glisodin (Salbu Melon), all measured by flexi Vent in response to increasing doses of the bronchoconstrictor methacholine. ANOVA analysis of the data using the Tukey test was carried out and a confidence interval as indicated on the charts established: *=p<0.05, ** p<0.01 and *** p<0.001, in a population of n=6 to 8 mice per group.

### Part 5—Inflammatory Response

At the end of the study protocol on mice, bronchoalveolar lavage (BAL) were taken and the cell population analysed using flow cytometry. The results of this analysis are illustrated in FIG. 9, which shows the total number of cells (Total), the number of Ly6C+ neutrophils (Neu), the number of CCR3+ neutrophils (Eo), the number of Ly6G+ monocytes (Mo) and CD3+ lymphocytes (Ly) where CTI, are naïve untreated mice, CTI wGli are naïve mice having received wheat Glisodin, CTLmGli are naïve mice having received melon Glisodin, HDM are asthmatic mice without treatment, HDMwGli are asthmatic mice having received wheat Glisodin, HDMmGli are asthmatic mice having received melon Glisodin, Salbutamol are asthmatic mice having received a salbutamol treatment, Salbu Wheat are asthmatic mice having received a combined salbutamol/wheat Glisodin treatment, and Salbu Melon are asthmatic mice having received a combined salbutamol/melon Glisodin treatment. ANOVA analysis of the data using the Tukey test was carried out and a confidence interval as indicated on the charts established: *=p<0.05, ** p<0.01 and *** p<0.001, in a population of n-6 to 8 mice per group.

The total number of cells in the BAL and lungs had significantly increased in the asthmatic mice compared to the control mice. Nonetheless, this number was significantly reduced in those mice having received wheat or melon Glisodin, in comparison to untreated asthmatic mice, cf. FIGS. 9A and 9C. The reduction in cell number is most visible in eosinophils and lymphocytes, and to a lower extent, in monocytes, but not neutrophils. In common with the respiratory function analysis, no significant difference in cell number reduction was observed when comparing wheat and melon Glisodin side-by-side. A test for any cumulative or synergistic effect of Glisodin administration combined with salbutamol administration was also carried out, and the results illustrated in FIGS. 9B and 9D. Salbutamol showed a characteristic anti-inflammatory effect, resulting in a reduction of the number of cells in the BAL, and in particular in the number of eosinophils and lymphocytes. No complementary effect was observed in the combined treatment administrations of either wheat or melon Glisodin, cf. FIGS. 9B and 9D.

### Part 6—Immune Response

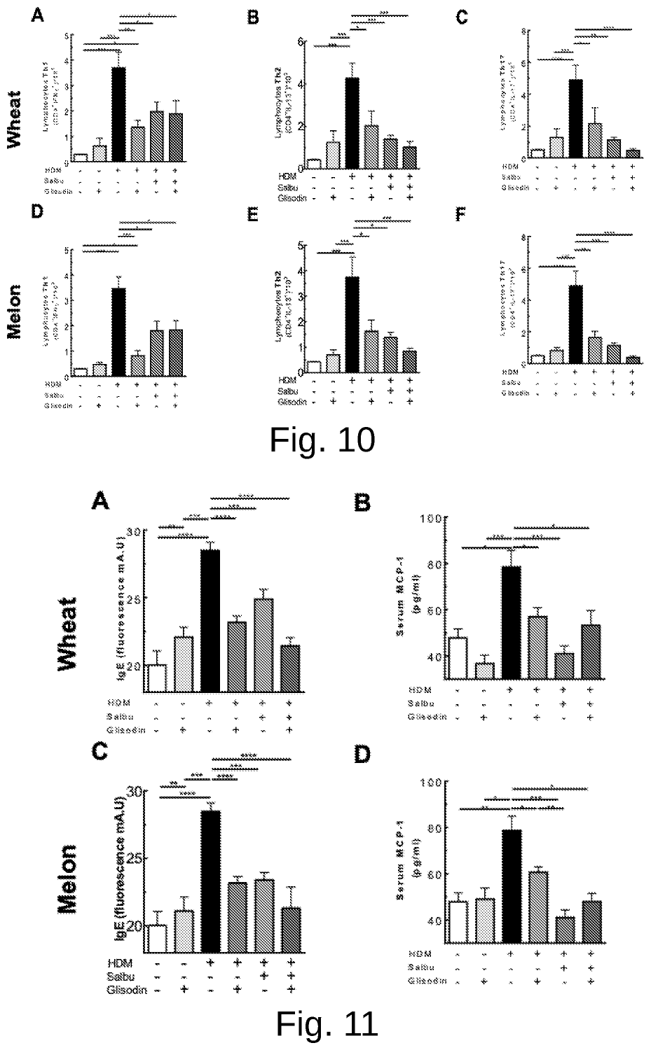

At the end of the treatment protocol, mice lungs were harvested, ground, restimulated with whole dust mite extract and the resulting cell populations analysed using flow cytometry. The results are illustrated in FIG. 10, in which the following cell types were counted: Th1 (CD3+, CD4+, IFNγ+), Th2 (CD3+, CD4+, IL-13+) and Th17 (CD3+, CD4+, IL-17+). ANOVA analysis of the data using the Tukey test was carried out and a confidence interval as indicated on the charts established: *=p<0.05, ** p<0.01 and *** p<0.001, in a population of n=6 to 8 mice per group.

T lymphocyte differentiation is a phenomena in which a naïve T lymphocyte, having a given specificity for an antigen, also acquires particular effect capabilities. This process is induced in the lymph ganglions during interaction between a cell that presents with the specific antigen and a naïve T lymphocyte. Dependent on the environment in which it has been activated before migrating to the lymph organs, these antigen presenting cells produce a number of different cytokines which can induce a number of different differentiation programs for the T lymphocytes. As a result, there are a number of routes for T lymphocyte differentiation, which are more less mutually exclusive. In this way, the various different populations of T lymphocytes can be distinguished by the type and distribution of the cytokines produced. In summary, these are presented as follows:
Th1 lymphocytes which produce γ-IFN, and which are involved in the antiviral and antibacterial response;
Th2 lymphocytes which produce mostly IL-4 and IL-13, which are involved in the antiparasitic response and in the allergic response;
Th17 lymphocytes which produce IL-17, which are involved in the inflammatory response.

From FIG. 10 it can be seen that Th1, Th2 and Th17 lymphocyte cell numbers are all significantly higher in asthmatic mice when compared to control mice. In contrast thereto, asthmatic mice having received Glisodin show a marked reduction in Th1 lymphocyte numbers, cf. FIGS. 10A and 10D. Similarly, mice that were treated with salbutamol, whether or not in combined treatment with Glisodin, also showed a reduction in Th1 lymphocyte numbers, although no cumulative effect was observed, cf. FIGS. 10A et 10D. No noticeable difference was observed between the two Glisodin formulations. Similar results were observed with regard to the Th2 lymphocyte response (cf. FIGS. 10B and 10 E) and Th17 lymphocyte response (cf. FIGS. 10C and 10F) with a tendency of cumulative effect between Glisodin and salbutamol.

### Part 7—Humoral Response

In allergic asthma, once the antigen has been recognised, clonal selection and proliferation taken place, followed by B lymphocyte differentiation into plasmocytes, the formation of the immune complex between IgE and allergens leads to degranulation of the effecting cells and liberation of chemical mediators such as histamine and MCP-1. In order to evaluate the effect of Glisodin treatment, with respect to initiation of an asthma attack, serum levels of IgE were measured by ELISA for IgE specific to house dust mites and measurements made of the levels of production of MCP-1. The results of these measurements are illustrated in FIG. 11, with application of ANOVA analysis of the data using the Tukey test leading to a confidence interval as indicated on the charts established: *=p<0.05, ** p<0.01 et *** p<0.001, in a population of n=6 to 8 mice per group.

The level of HDM-specific IgE was higher in the asthmatic mice than in the control mice, and it was further observed that both salbutamol and Glisodin were able to reduce those levels, without any indication of a cumulative effect, cf. FIGS. 11A and 11C. Similarly, on initiation of an asthma attack, as characterised by the production of MCP-1, a greater level of this molecule is observed in asthmatic mice than in the control mice, cf. FIGS. 11B and 11D. However, contrary to the results presented for the other analyses, in this case salbutamol reduced the level of MCP-1 produced, whereas Glisodin did not seem to be as effective, cf. FIGS. 11B and 11D.

### Part 8—Cytokine Response

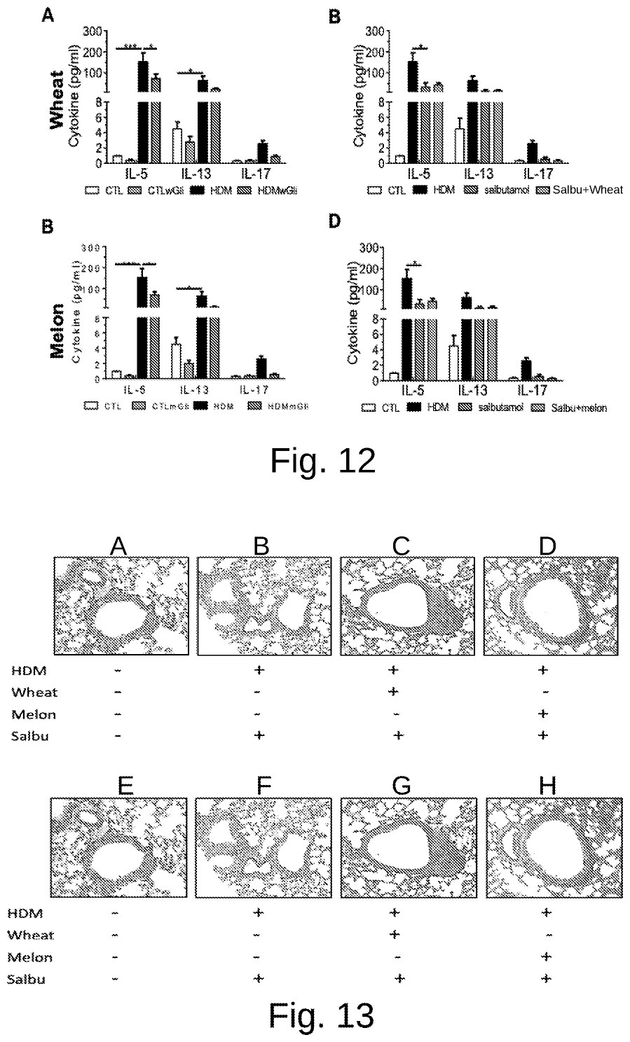

Cytokines are produced in response to antigens present at the surface of foreign bodies or by molecules considered as foreign by the immune. Once an antigen response has been initiated, cells which are entrusted with the development of the immune defence are activated, and notably, result in stimulation of growth and differentiation of lymphocytes. In the present case, Th2 inflammatory pathway cytokines, for example, IL-5 and IL-13, and Th17 cytokines, such as IL-17 were measured in the supernatant of BAL of the various mouse groups, and the results illustrated in FIG. 12, with application of ANOVA analysis of the data using the Tukey test leading to a confidence interval as indicated on the charts established: *=p<0.05, ** p<0.01 and *** p<0.001, in a population n=6 to 8 mice per group.

As illustrated in FIG. 12, an increase in cytokine count was observed for asthmatic mice in comparison to control mice. Glisodin formulation and salbutamol were effective in reducing the IL-5 count and have a tendency to decrease IL-17 count. However, it was also observed that neither salbutamol nor the various Glisodin formulations tested were able to reduce the level of IL-13 (FIGS. 12A to 12D).

### Part 9—Tissular Degradation

In situ evaluation of inflammation, bronchoconstriction and asthma severity were carried out via histological analysis, through staining of lung samples from the various mouse groups with eosin and hematoxylin, with a population of 6 mice per group, the images being shown at a scale of 100 μm, as illustrated in FIG. 13. The degree of tissular degradation was assessed in a double blind protocol by two independent assessors using a scoring system. A reduction in the severity of the asthma was observed in mice treated with Glisodin or with salbutamol, but no significant cumulative effect of both treatments together was observed. Additionally, no noticeable difference between the two Glisodin products was observed either.

The conclusion to be drawn from the various analyses carried out and described above is that interestingly, Glisodin prevented the development of airway hyperresponsiveness in asthma and suppressed airway eosinophilia in allergic mice. Moreover, it prevented the increase in IL-5 and diminished the HDM induced Th2 and Th17 response in the lungs of allergic mice. the administration of Glisodin leads to an improvement in respiratory function in asthmatic mice. This effect is linked to an anti-inflammatory effect vis-à-vis the immune response, and a corresponding reduction in the humoral response. Whilst Glisodin administration did not appear to prevent exacerbation of the asthmatic condition, it did have a positive effect on allergic sensitisation. These conclusions were furthermore confirmed during histological analysis.

1. Use of a superoxide dismutase for the treatment and/or prevention of allergic asthma in mammals, wherein the superoxide dismutase is administered orally alone, or optionally in combination with the administration of an aerosol preparation comprising at least one ß₂-adrenergic receptor agonist.

2. Use according to claim 1, wherein the mammal is a human.

3. Use according to claim 1, wherein the superoxide dismutase is a heterologous superoxide dismutase, preferably a plant-derived superoxide dismutase.

4. Use according to claim 1, wherein the plant-derived superoxide dismutase is selected from the group consisting of melon superoxide dismutase, and wheat superoxide dismutase.

5. Use according to claim 1, wherein the oral administration of superoxide dismutase consists of a solid-form preparation comprising a plant-derived superoxide dismutase in association with a prolamine.

6. Use according to claim 1, wherein the oral administration of superoxide dismutase consists of a solid-form preparation comprising a plant derived superoxide dismutase in association with gliadin.

7. Use according to claim 1, wherein the oral administration of superoxide dismutase consists of a solid-form preparation comprising:
a) from 0.50% to 5.00%, by total weight of the solid form preparation, of a dried melon juice-derived extract containing a standardized titer of superoxide dismutase;
b) from 1.50% to 5.00%, by total weight of the preparation, of gliadin; and
c) qsp maltodextrin,
d) wherein the superoxide dismutase has an enzymatic activity comprised between 1 to 5 IU/mg.

8. Use according to claim 1, wherein the oral administration of superoxide dismutase consists of a solid-form preparation comprising:
e) 1.33% by total weight of the solid form preparation, of a dried melon juice-derived extract containing a standardized titer of superoxide dismutase;
f) 3.33%, by total weight of the preparation, of gliadin; and
g) qsp maltodextrin,
h) wherein the superoxide dismutase has an enzymatic activity of 1.5 U (NBT)/mg.

9. Use according to claim 1, wherein the oral administration of superoxide dismutase consists of a solid-form preparation comprising:
i) from 0.04% to 0.40%, by total weight of the preparation, of a wheat-derived superoxide dismutase;
j) from 1.50% to 5.00%, by total weight of the preparation, of gliadin; and
k) qsp maltodextrin,
1, wherein the superoxide dismutase has an enzymatic activity comprised between 1 to 10 U (NBT)/mg.

10. Use according to claim 1, wherein the oral administration of superoxide dismutase consists of a solid-form preparation comprising:
m) 0.18%, by total weight of the preparation, of a wheat-derived superoxide dismutase;
n) 3.50%, by total weight of the preparation, of gliadin; and
o) qsp maltodextrin,
p) wherein the superoxide dismutase has an enzymatic activity of 4.5 U (NBT)/mg.

11. Use according to claim 1, wherein the superoxide dismutase is administered orally to a human in an amount comprised between 100 mg to 2000 mg per day, continuously for a minimum period of 15 days.

12. Use according to claim 11, wherein the superoxide dismutase is administered orally to a human in an amount of 500 mg per day, continuously for a minimum period of 15 days, and up to 180 days.

13. Use according to claim 1, wherein the at least one ß2-adrenergic receptor agonist is selected from the group consisting of bitolterol, fenoterol, isoprenaline, levosalbutamol, orciprenaline, pirbuterol, procaterol, ritodrine, salbutamol, and terbutaline.

14. Use according to claim 1, wherein the at least one ß₂-adrenergic receptor agonist is salbutamol.
